(19) United States
(12) Patent Application Publication
(10) Pub. No.: US 2005/0020517 A1
(43) Pub. Date: Jan. 27, 2005

(54) Treatment of HIV infection through combined administration of tipranavir and capravirine

(75) Inventors: Ulrich Walter Drees, Ingelheim (DE); Douglas Lytle Mayers, Newtown, CT (US); Scott McCallister, Westport, CT (US)

Correspondence Address: BOEHRINGER INGELHEIM CORPORATION, 900 RIDGEBURY ROAD, P O BOX 368, RIDGEFIELD, CT, 06877, US

(73) Assignee: Boehringer Ingelheim Pharmaceuticals, Inc., Ridgefield, CT (US)

(21) Appl. No.: 10/720,550

(22) Filed: Nov. 24, 2003

Related U.S. Application Data
(60) Provisional application No. 60/433,679, filed on Dec. 16, 2002.

(51) Int. Cl. A61K 31/7056; A61K 31/522

(52) U.S. Cl. 514/43; 514/263.32

(57) ABSTRACT

An improved method for treating HIV infection comprising administering to a human in need of such treatment a combination of a therapeutically effective amount of tipranavir or a pharmaceutically acceptable salt thereof, and a therapeutically effective amount of capravirine or a pharmaceutically acceptable salt thereof.

### RELATED APPLICATIONS

Benefit of U.S. Provisional Application Ser. No. 60/433,679 filed on Dec. 16, 2002 is hereby claimed.

### FIELD OF THE INVENTION

The present invention relates to an improved method for treating HIV infection comprising administering to a human in need of such treatment a combination of a therapeutically effective amount of tipranavir or a pharmaceutically acceptable salt thereof, and a therapeutically effective amount of capravirine or a pharmaceutically acceptable salt thereof.

### BACKGROUND OF THE INVENTION

Human immunodeficiency virus (HIV) is recognized as the causative agent in AIDS.

Current therapies for HIV infection focus on inhibiting the activity of viral enzymes which are essential to the life cycle of the virus. The agents that are presently in use fall mainly into three classes, designated Nucleoside Reverse Transcriptase Inhibitors (NRTIs), Non-nucleoside Reverse Transcriptase Inhibitors (NNRTIs), and Protease Inhibitors (PIs). Presently, combination therapies, i.e. the selection of two or more antiretroviral agents taken together to make up a “drug cocktail,” are the preferred treatment for HIV infection. Combination therapies have been shown to reduce the incidence of opportunistic infections and to increase survival time. Typically, the drug cocktail combines drugs from different classes, so as to attack the virus at several stages in the replication process. This approach has been shown to reduce the likelihood of the development of virus forms that are resistant to a given drug or class of drugs.

Treatment failure with rebound of the amount of HIV which can be measured in the blood is common for patients treated with combination antiretroviral regimens. Resistance to the drugs in the drug regimen develops as the virus replicates in the presence of these drugs. Because of structural similarities of the drugs within an antiretroviral class, cross resistance is commonly seen to the other members of that class (for example virologic failure on a regimen containing an NNRTI will lead to cross resistance to the other first generation NNRTI agents). As patients experience repeated virologic failure on antiretroviral combination therapy, their viruses develop broad multi-class antiretroviral drug resistance which limits the effectiveness of the next round of antiretroviral therapy. Many highly treatment experienced patients have been exposed to all three classes of antiretroviral drugs and cannot obtain two active drugs to form the core of a new, effective antiretroviral drug regimen.

Tipranavir and capravirine are both known agents for the treatment of HIV infection.

Tipranavir, also known as U-140690 and PNU-140690, is an HIV protease inhibitor. Chemically, tipranavir is (6R)-3-((1R)-1-[3-({[5-trifluoromethyl)(2-pyridyl)]sulfonyl}amino)phenyl]propyl}-4-hydroxy-6-(2-phenylethyl)-6-propyl-5,6-dihydro-2H-pyran-2-one or ([R—(R*,R*)]-N-[3-[1-[5,6-dihydro-4-hydroxy-2-oxo-6-(2-phenylethyl)-6-prop yl-2H-pyran-3-yl]propyl]phenyl]-5-(trifluoromethyl)-2-pyridinesulfonamide). It has the following structural formula:

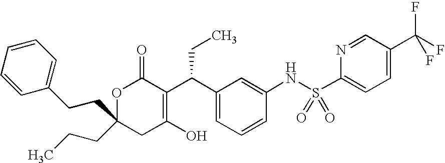

Tipranavir, and methods for its synthesis and use in the treatment of HIV are described in WO 95/30670 and corresponding U.S. Pat. No. 5,852,195. Pharmaceutical formulations suitable for the oral administration of tipranavir are described in WO 99/06043 and WO 99/06044, and the corresponding U.S. Pat. Nos. 6,121,313 and 6,231,887.

As tipranavir is metabolized relatively rapidly by the the cytochromes P450, especially the Cyp3A4 isoform, it is necessary to co-administer an inhibitor of Cyp3A4 in order to obtain therapeutically effective blood levels of tipranavir. The use of ritonavir for this purpose is described in U.S. Pat. No. 6,147,095. The use for this purpose of other inhibitors of Cyp3A4 is possible.

Capravirine (CAS REGISTRY NUMBER 178979-85-6), also known as AG 1549 and S 1153, is an HIV NNRTI. Chemically, capravirine is, 5-[(3,5-dichlorophenyl)thio]-4-(1-methylethyl)-1-(4-pyridinylmethyl)-1H-imidazole-2-methanol carbamate ester. It has the following structural formula:

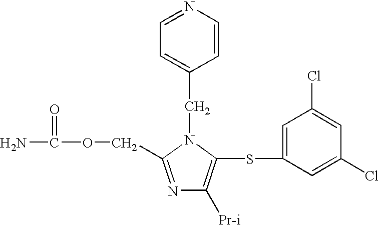

Capravirine, methods for its synthesis and use in the treatment of HIV, and pharmaceutical preparations suitable for carrying out such treatment, are described in U.S. Pat. No. 5,910,506.

### SUMMARY OF THE INVENTION

The present invention provides an improved method for treating highly treatment experienced HIV-infected patients. The method comprises administering tipranavir in combination with an inhibitor of Cyp3A4 (such as ritonavir), in further combination with capravirine and an optimized background regimen of nucleoside reverse transcriptase inhibitors to provide an active antiretroviral regimen.

### DETAILED DESCRIPTION OF THE INVENTION

The present invention provides an improved method for treating HIV infection, especially in highly treatment experienced HIV-infected patients. The method comprises administering tipranavir in combination with an inhibitor of Cyp3A4, in further combination with capravirine and an optimized background regimen of nucleoside reverse transcriptase inhibitors to provide an active antiretroviral regimen that is particulary well suited for the treatment of highly treatment experienced HIV-infected patients.

As used herein, the term “highly treatment experienced HIV-infected patients” means HIV-infected patients with virologic failure (detectable HIV RNA in their blood) who have previously received treatment with 2 or more combination antiretroviral regimens.

As used herein, the term “optimized background regimen of nucleoside reverse transcriptase inhibitors” means a combination of nucleoside or nucleotide agents selected on the basis of all of the information available to the treating provider including drug history, knowledge of resistance/cross resistance and, where available, genotypic or phenotypic drug resistance test results for the virus in the HIV-infected patient's blood.

When employed in accordance with the method of the invention, tipranavir can be administered in the manner described in WO 95/30670 and corresponding U.S. Pat. No. 5,852,195. It is preferred to administer this substance orally using pharmaceutical formulations such as those described in WO 99/06043 and WO 99/06044, and the corresponding U.S. Pat. Nos. 6,121,313 and 6,231,887. It is particularly preferred to administer the tipranavir at the dosage of 500 to 750 mg PO BID.

A suitable inhibitor of Cyp3A4 is ritonavir, which can be administered as described in U.S. Pat. No. 6,147,095. For the purposes of the present invention it is preferred to administer ritonavir at a dosage of 200 mg PO BID. The use for this purpose of other inhibitors of Cyp3A4 is also possible.

When used in accordance with the present invention, capravirine is administered in the manner described in U.S. Pat. No. 5,910,506. The preferred dosage of capravirine will range from 400 to 1400 mg PO BID.

Concomitantly administered nucleoside and nucleotide reverse transcriptase inhibitors will be given at standard doses, in the manner known to those of routine skill in the clinical treatment of HIV infection.

1. An improved method for treating highly treatment experienced HIV-infected patients, which method comprises administering tipranavir in combination with an inhibitor of Cyp3A4.

2. The method of claim 1 wherein the inhibitor of Cyp3A4 is ritonavir.

3. The method of claim 1 wherein the tipranavir, inhibitor of Cyp3A4 and capravirine are administered in further combination with an optimized background regimen comprising at least one nucleoside reverse transcriptase inhibitor.
